(19) United States
(12) Patent Application Publication
(10) Pub. No.: US 2026/0137459 A1
(43) Pub. Date: May 21, 2026

(54) INFORMATION PROCESSING APPARATUS, ULTRASONIC DIAGNOSTIC APPARATUS, INFORMATION PROCESSING METHOD, AND NON-TRANSITORY COMPUTER-READABLE STORAGE MEDIUM STORING INFORMATION PROCESSING PROGRAM

(71) Applicant: KONICA MINOLTA, INC., Tokyo (JP)

(72) Inventor: Kazuya TAKAGI, Tokyo (JP)

(21) Appl. No.: 19/393,782

(22) Filed: Nov. 19, 2025

(30) Foreign Application Priority Data
Nov. 21, 2024 (JP) 2024-202904

(51) Int. Cl. A61B 34/20; A61B 8/00; A61B 8/08

(52) U.S. Cl. CPC A61B 34/20; A61B 8/0841; A61B 8/461; A61B 2034/2063

(57) ABSTRACT

An information processing apparatus includes a hardware processor that acquires an ultrasonic image of a subject into which a needle-shaped instrument has been inserted, and outputs an estimation result that is output by inputting the ultrasonic image acquired into a machine learning model. The machine learning model is trained on a data set of a training ultrasonic image in which at least a part of a needle-shaped instrument is captured and region information corresponding to a two-dimensional region in which the needle-shaped instrument is present in the training ultrasonic image.

### CROSS REFERENCE TO RELATED APPLICATION

The present invention claims priority under 35 U.S.C. § 119 to Japanese patent application No. 2024-202904, filed on Nov. 21, 2024, the entire content of which is incorporated herein by reference.

### BACKGROUND OF THE INVENTION

### 1. Technical Field

The present invention relates to an information processing apparatus, an ultrasonic diagnostic apparatus, an information processing method, and a non-transitory computer-readable storage medium storing an information processing program.

### 2. Description of Related Art

Ultrasonic diagnostic apparatuses that generate ultrasonic images are used in medical fields and the like. Such an ultrasonic diagnostic apparatus includes an ultrasonic probe. A doctor or the like brings the ultrasonic probe into contact with the body surface of a subject and performs transmission and reception of ultrasonic waves with the ultrasonic probe. This makes it possible to acquire the shape and the like of tissues inside the body of the subject as an ultrasonic image.

Puncturing is sometimes performed on a subject in hospitals and the like. An ultrasonic diagnostic apparatus is sometimes used at the time of this puncturing. In orthopedic surgery, anesthesiology, pain clinic, dialysis, and the like, a doctor or the like uses an ultrasonic diagnostic apparatus to perform puncturing while checking the positional relationship between various tissues inside the body of a subject and a puncture instrument. Japanese Unexamined Patent Application Publication No. 2012-70837 discloses a technique for intelligibly displaying the position of a puncture instrument in an ultrasonic image.

### SUMMARY OF THE INVENTION

It is therefore desirable to be able to more accurately identify the position of the puncture instrument in the ultrasonic image.

The present invention has been made in view of the above circumstances, and an object thereof is to provide an information processing apparatus, an ultrasonic diagnostic apparatus, an information processing method, and a non-transitory computer-readable storage medium storing an information processing program, which are capable of more accurately identifying the position of a puncture instrument in an ultrasonic image.

To achieve at least one of the abovementioned objects, according to an aspect of the present invention,
an information processing apparatus reflecting one aspect of the present invention comprises the followings.

An information processing apparatus including a hardware processor that acquires an ultrasonic image of a subject into which a needle-shaped instrument has been inserted; and outputs an estimation result that is output by inputting the ultrasonic image acquired into a machine learning model, in which the machine learning model is trained on a data set of a training ultrasonic image in which at least a part of the needle-shaped instrument is captured and region information corresponding to a two-dimensional region in which the needle-shaped instrument is present in the training ultrasonic image.

### BRIEF DESCRIPTION OF THE DRAWINGS

Advantages and features provided by one or more embodiments of the present invention will be fully understood in conjunction with the following detailed description and the accompanying drawings. It should be however understood that these embodiments are merely illustrative and are not intended to limit the present invention.

FIG. 1 is a diagram illustrating the entire configuration of an ultrasonic diagnostic apparatus;

FIG. 2 is a block diagram illustrating a schematic configuration of an image processing apparatus illustrated in FIG. 1;

FIG. 3 is a block diagram illustrating a functional configuration of the image processing apparatus illustrated in FIG. 1;

FIG. 4 is a diagram for describing a training ultrasonic image and the like that are input into a machine learning model used in an estimator illustrated in FIG. 3;

FIG. 5 is a diagram illustrating an example of an estimation result displayed on a display illustrated in FIG. 2;

FIG. 6 is a diagram illustrating another example of the estimation result displayed on the display illustrated in FIG. 2;

FIG. 7 is a diagram illustrating another example of the estimation result displayed on the display illustrated in FIG. 2;

FIG. 8 is a diagram illustrating another example of the estimation result displayed on the display illustrated in FIG. 2;

FIG. 9 is a flowchart illustrating a procedure of estimation processing executed by the image processing apparatus illustrated in FIG. 1;

FIG. 10 is a flowchart illustrating a machine learning method of a trained model used in the estimation processing illustrated in FIG. 9;

FIG. 11 is a block diagram illustrating a functional configuration of an image processing apparatus according to a first modification example;

FIG. 12 is a diagram illustrating an example of a second mode displayed on a display of the image processing apparatus illustrated in FIG. 11;

FIG. 13 is a flowchart illustrating a procedure of estimation processing executed by the image processing apparatus illustrated in FIG. 11;

FIG. 14 is a block diagram illustrating a functional configuration of an image processing apparatus according to a second modification example;

FIG. 15 is a diagram illustrating an example of a puncture path displayed on a display of the image processing apparatus illustrated in FIG. 14;

FIG. 16 is a flowchart illustrating a procedure of estimation processing executed by the image processing apparatus illustrated in FIG. 14;

FIG. 17 is a block diagram illustrating a functional configuration of an image processing apparatus according to a third modification example;

FIG. 18 is a flowchart illustrating a procedure of estimation processing executed by the image processing apparatus illustrated in FIG. 17;

FIG. 19 is a block diagram illustrating a functional configuration of an image processing apparatus according to a fourth modification example;

FIG. 20 is a diagram for describing a data set used by a tissue predictor illustrated in FIG. 19;

FIG. 21 is a diagram illustrating an example of a target tissue displayed on a display of the image processing apparatus illustrated in FIG. 19;

FIG. 22 is a flowchart illustrating a procedure of estimation processing executed by the image processing apparatus illustrated in FIG. 19;

FIG. 23 is a block diagram illustrating a functional configuration of an image processing apparatus according to a fifth modification example;

FIG. 24 is a diagram illustrating an example of a Doppler image displayed on a display of the image processing apparatus illustrated in FIG. 23; and

FIG. 25 is a flowchart illustrating a procedure of estimation processing executed by the image processing apparatus illustrated in FIG. 23.

### DETAILED DESCRIPTION

Hereinafter, one or more embodiments of the present invention will be described with reference to the drawings. However, the scope of the invention is not limited to the disclosed embodiments.

### EMBODIMENT

### <Configuration of Ultrasonic Diagnostic Apparatus 1>

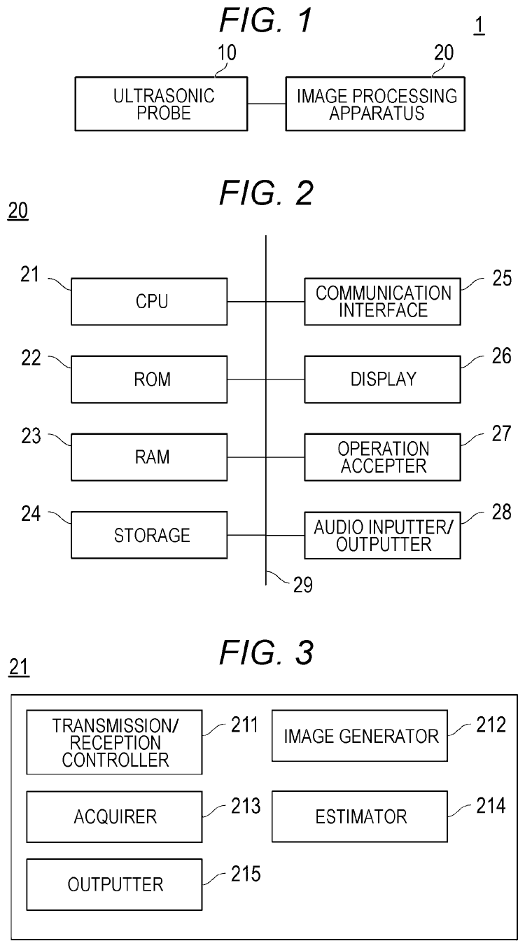

FIG. 1 illustrates a schematic configuration of an ultrasonic diagnostic apparatus 1 according to an embodiment. The ultrasonic diagnostic apparatus 1 includes an ultrasonic probe 10 and an image processing apparatus 20 connected to the ultrasonic probe 10. The ultrasonic diagnostic apparatus 1 is used when a doctor, a clinical engineer, or the like punctures a subject in a hospital or the like. That is, a user of the ultrasonic diagnostic apparatus 1 is a doctor or the like. Here, the ultrasonic probe 10 corresponds to a specific example of a probe of the present invention, and the image processing apparatus 20 corresponds to a specific example of an information processing apparatus of the present invention.

The user brings the ultrasonic probe 10 into contact with the body surface of a subject and causes the ultrasonic diagnostic apparatus 1 to generate an ultrasonic image of the inside of the body of the subject. The user moves a puncture instrument inside the body of the subject while checking the ultrasonic image displayed on a display of the image processing apparatus 20. The puncture instrument is a puncture needle or the like.

The ultrasonic probe 10 irradiates the inside of the body of the subject with ultrasonic waves from the body surface of the subject, and receives the ultrasonic waves reflected inside the body of the subject. Transmission and reception of the ultrasonic waves by the ultrasonic probe 10 are controlled by the image processing apparatus 20. The ultrasonic probe 10 converts the received ultrasonic waves into electrical signals and transmits the electrical signals to the image processing apparatus 20. The ultrasonic waves have frequencies in the range of about 1 MHz to about 30 MHz. The ultrasonic probe 10 includes a linear probe, a convex probe, a sector probe, a three-dimensional probe, or the like.

The image processing apparatus 20 generates an ultrasonic image of the inside of the body of the subject on the basis of the electrical signals received from the ultrasonic probe 10. The image processing apparatus 20 is a medical image processing apparatus. The image processing apparatus 20 includes, for example, a computer such as a PC. PC is an abbreviation for “Personal Computer”. The image processing apparatus 20 may include a smartphone, a tablet terminal, or the like.

FIG. 2 is a block diagram illustrating a schematic configuration of the image processing apparatus 20. The image processing apparatus 20 includes a CPU 21, a ROM 22, a RAM 23, a storage 24, a communication interface 25, a display 26, an operation accepter 27, and an audio inputter/outputter 28. The components are communicably connected to each other via a bus 29. CPU is an abbreviation for “Central Processing Unit”. ROM is an abbreviation for “Read Only Memory”. RAM is an abbreviation for “Random Access Memory”. Here, the storage 24 corresponds to a specific example of a memory of the present invention. At least one of the display 26 and the audio inputter/outputter 28 corresponds to a specific example of a notificator of the present invention.

The CPU 21 controls the above-described components and performs various kinds of arithmetic processing in accordance with programs recorded in the ROM 22 or the storage 24. Specific functions of the CPU 21 will be described later.

The ROM 22 stores various programs and various pieces of data.

The RAM 23, as a workspace, temporarily stores programs and data.

The storage 24 stores various programs including an operating system and various pieces of data. The storage 24 has installed therein an application for estimating, from an ultrasonic image of the inside of the body of a subject, the position of a puncture instrument within the ultrasonic image, using a trained machine learning model. The storage 24 may store a plurality of ultrasonic images. The storage 24 may store information regarding the subject such as the name of the subject. Furthermore, the storage 24 may store a trained machine learning model, a data set to be used for machine learning, and the like.

The communication interface 25 is an interface for communicating with other devices. As the communication interface 25, a communication interface based on various wired or wireless standards is used. The communication interface 25 is used for transmission of signals to the ultrasonic probe 10, reception of signals from the ultrasonic probe 10, and the like. The communication interface 25 may be used for connection between the image processing apparatus 20 and an external device such as an external server.

The display 26 includes a liquid crystal display, an organic EL display, or the like. EL is an abbreviation for “Electroluminescence”. Various kinds of information are displayed on the display 26. The display 26 may be constituted by viewer software, a printer, or the like.

The operation accepter 27 includes a touch sensor, a pointing device, a keyboard, or the like. The pointing device is a mouse or the like. The operation accepter 27 accepts various user operations. The display 26 and the operation accepter 27 may constitute a touch screen.

The audio inputter/outputter 28 includes a speaker, a microphone, and the like.

### <Functions of Image Processing Apparatus 20>

FIG. 3 is a block diagram illustrating a functional configuration of the image processing apparatus 20. The image processing apparatus 20 functions as a transmission/reception controller 211, an image generator 212, an acquirer 213, an estimator 214, and an outputter 215 when the CPU 21 reads a program stored in the storage 24 and executes processing.

The transmission/reception controller 211 controls the irradiation of ultrasonic waves and the reception of ultrasonic waves by the ultrasonic probe 10. The transmission/reception controller 211 transmits a voltage pulse to the ultrasonic probe 10, and controls the irradiation of ultrasonic waves from the ultrasonic probe 10 to the subject. The transmission/reception controller 211 adjusts the voltage amplitude, the pulse width, and the transmission timing of the voltage pulse, and transmits the voltage pulse for each channel of the ultrasonic probe 10. The transmission/reception controller 211 sets an appropriate delay time for each of the plurality of channels. As a result, the depth of the position irradiated with the ultrasonic waves and the pulse waveform are adjusted.

The transmission/reception controller 211 receives electrical signals corresponding to the ultrasonic waves received by the ultrasonic probe 10. The transmission/reception controller 211 amplifies the electrical signals received for the respective channels and converts the resultant electrical signals into digital signals. The transmission/reception controller 211 may perform phasing addition on the signals received for the respective channels.

The image generator 212 acquires the signals from the transmission/reception controller 211 and generates an ultrasonic image of the inside of the body of the subject. The ultrasonic image of the inside of the body of the subject is a tomographic image. The image generator 212 generates an ultrasonic image of a cross section including a transmission direction and a scanning direction of ultrasonic waves by the ultrasonic probe 10.

The acquirer 213 acquires the ultrasonic image generated by the image generator 212. This ultrasonic image is an ultrasonic image of the subject into which a puncture instrument has been inserted. The acquirer 213 may acquire image information regarding the ultrasonic image.

The estimator 214 estimates a puncture instrument region in which the puncture instrument is present in the ultrasonic image acquired by the acquirer 213. The estimator 214 estimates a puncture instrument region by inputting the ultrasonic image into a machine learning model. This machine learning model is trained on a data set of a training ultrasonic image in which at least a part of the puncture instrument is captured and region information corresponding to a two-dimensional region in which the puncture instrument is present in this training ultrasonic image. The machine learning model is trained on a plurality of data sets.

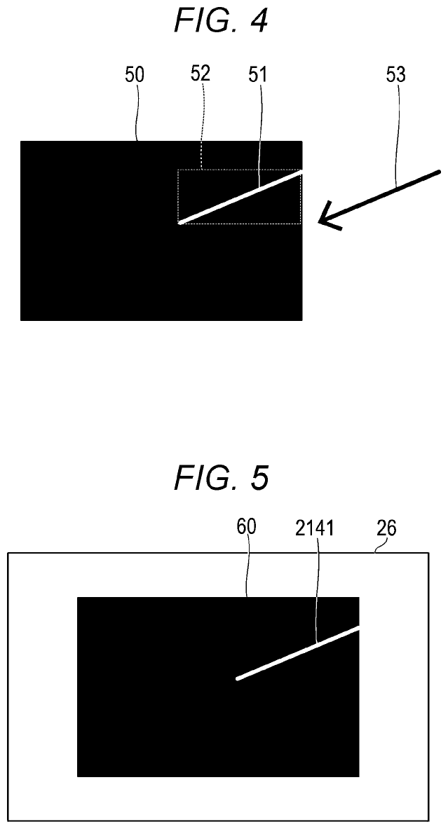

FIG. 4 illustrates an example of the relationship between a training ultrasonic image 50 and a two-dimensional region 52 in the training ultrasonic image 50. The training ultrasonic image 50 is a B-mode image. B-mode is an abbreviation for “Brightness mode”. The training ultrasonic image 50 captures a puncture instrument 51. The two-dimensional region 52 is a region in which the puncture instrument 51 is present in the training ultrasonic image 50, and has a rectangular shape. The two-dimensional region 52 is a partial area within the training ultrasonic image 50. The two-dimensional region 52 is a continuous region. In other words, the two-dimensional region 52 is not a point cloud. The two-dimensional region 52 contains portions having mutually-different degrees of brightness. The region information regarding the two-dimensional region 52 includes information regarding the position of the two-dimensional region 52 within the training ultrasonic image 50, information regarding the size of the two-dimensional region 52, information regarding the shape of the two-dimensional region 52, information regarding the brightness within the two-dimensional region 52, and the like.

The puncture instrument 51 having a needle shape is disposed on a diagonal line of the two-dimensional region 52. In other words, the two-dimensional region 52 is set such that the puncture instrument 51 is disposed on a diagonal line. It is preferable that the tip of the puncture instrument 51 is present in the two-dimensional region 52, and the tip of the puncture instrument 51 is disposed at a corner portion of the two-dimensional region 52 having a rectangular shape. The data set preferably further includes information regarding a puncture direction 53 of the puncture instrument 51 in the training ultrasonic image 50. The puncture direction 53 represents a direction toward the tip of the puncture instrument 51 within the two-dimensional region 52. In FIG. 4, the puncture direction 53 is a direction toward the lower left on the page. The data set may include information regarding a position at which the tip of the puncture instrument 51 is present within the two-dimensional region 52.

The outputter 215 outputs an estimation result from the estimator 214. This estimation result is obtained by inputting the ultrasonic image acquired by the acquirer 213 into the machine learning model. The estimation result includes information regarding the puncture instrument region estimated by the estimator 214. The outputter 215 outputs the estimation result by causing the display 26 to display the estimation result.

FIG. 5 illustrates an example of the estimation result displayed on the display 26. The display 26 displays an ultrasonic image 60 and a puncture instrument region 2141 within the ultrasonic image 60. The ultrasonic image 60 is an image generated by the image generator 212. The ultrasonic image 60 is a B-mode image. The puncture instrument region 2141 is a region of the ultrasonic image 60 in which the puncture instrument is estimated to be present. The puncture instrument region 2141 has a needle shape. The puncture instrument region 2141 is estimated by the estimator 214.

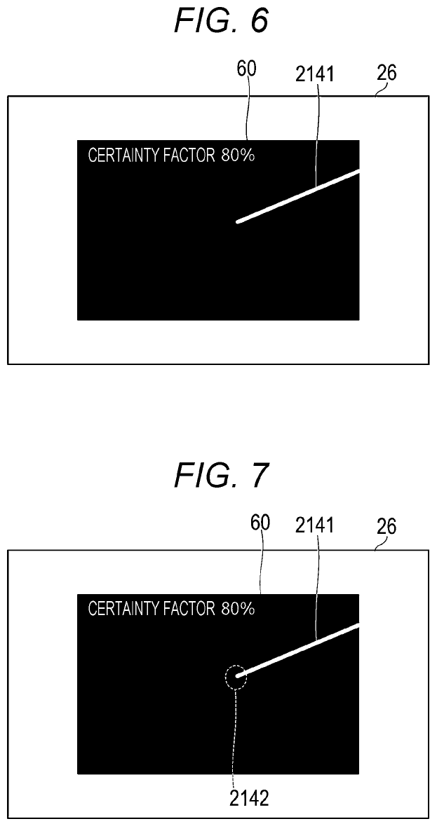

FIG. 6 to FIG. 8 each illustrate another example of the estimation result displayed on the display 26. As illustrated in FIG. 6 and the like, the estimation result may include information regarding a certainty factor of the puncture instrument region 2141 estimated by the estimator 214. The certainty factor is a value representing the plausibility of the estimated puncture instrument region 2141, and the larger the value of the certainty factor is, the higher the reliability of the estimated puncture instrument region 2141 is. The certainty factor is expressed in the range of 0% to 100%. When the estimator 214 inputs the ultrasonic image 60 into the machine learning model, the certainty factor is output together with the puncture instrument region 2141. The output of the information regarding the certainty factor makes it easier for the user to determine whether the puncture instrument is captured within the ultrasonic image 60.

As illustrated in FIG. 7, the estimation result may include information regarding a position 2142 at which the tip of the puncture instrument is present in the puncture instrument region 2141. In the display 26, the position 2142 is indicated by being surrounded in a circular shape. When the estimator 214 inputs the ultrasonic image 60 into the machine learning model, the information regarding the position 2142 at which the tip of the puncture instrument is present is output together with the puncture instrument region 2141. The output of the information regarding position 2142 makes it possible for the user to grasp the situation of the puncture instrument inside the body of the subject more accurately and to perform puncturing smoothly. The estimation result may include information regarding the puncture direction of the puncture instrument region 2141.

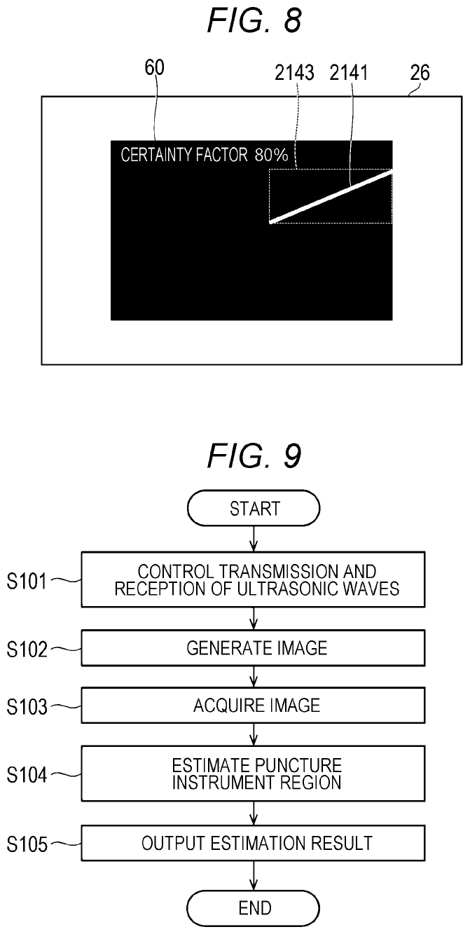

As illustrated in FIG. 8, the estimation result may include information regarding an estimation region 2143 including the puncture instrument region 2141. The estimation region 2143 is a region corresponding to the two-dimensional region 52 in the training ultrasonic image 50, and has a rectangular shape. The puncture instrument region 2141 is disposed on a diagonal line of the estimation region 2143. When the estimator 214 inputs the ultrasonic image 60 into the machine learning model, information regarding the puncture instrument region 2141 is output together with the estimation region 2143.

### <Outline of Processing Executed by Image Processing Apparatus 20>

FIG. 9 is a flowchart illustrating a procedure of puncture instrument region estimation processing executed by the image processing apparatus 20. The processing of the image processing apparatus 20 illustrated in the flowchart of FIG. 9 is stored as a program in the storage 24 of the image processing apparatus 20 and is executed by the CPU 21 controlling the respective units.

### (Step S101)

First, the image processing apparatus 20 controls transmission and reception of ultrasonic waves by the ultrasonic probe 10. The ultrasonic probe 10 irradiates the inside of the body of the subject with ultrasonic waves from the body surface of the subject, and receives the ultrasonic waves reflected inside the body of the subject. The ultrasonic probe 10 converts signals regarding the ultrasonic waves into electrical signals and transmits the electrical signals to the image processing apparatus 20. A puncture instrument has been inserted into the body of the subject.

### (Step S102)

The image processing apparatus 20 generates the ultrasonic image 60 on the basis of the electrical signals transmitted from the ultrasonic probe 10 in step S101.

### (Step S103)

The image processing apparatus 20 acquires the ultrasonic image 60 generated in step S102.

### (Step S104)

The image processing apparatus 20 estimates the puncture instrument region 2141 by inputting the ultrasonic image 60 acquired in step S103 into the machine learning model. At this time, the image processing apparatus 20 calculates the certainty factor of the puncture instrument region 2141.

### (Step S105)

The image processing apparatus 20 outputs the estimation result estimated in step S104. This estimation result includes the information regarding the puncture instrument region 2141 and the information regarding the certainty factor of the puncture instrument region 2141. The image processing apparatus 20 causes the display 26 to display the ultrasonic image 60, the puncture instrument region 2141, and the certainty factor. The image processing apparatus 20 executes this estimation processing over time. This makes it possible for the user to accurately grasp the position of the puncture instrument moving inside the body of the subject.

### <Learning Processing>

Next, a machine learning method of the machine learning model used for estimation of puncture instrument region 2141 will be described.

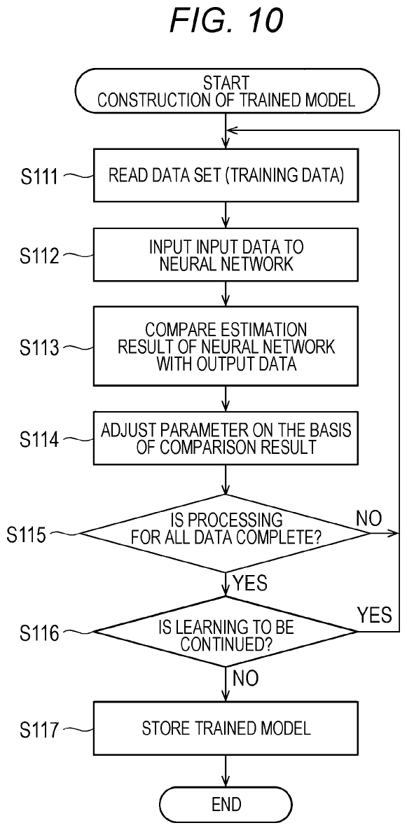

FIG. 10 is a flowchart illustrating a machine learning method of a machine learning model.

In the processing in FIG. 10, machine learning is executed by using a large number of data sets prepared in advance as training sample data. In the data sets, the training ultrasonic image 50 is the input, and the region information regarding the two-dimensional region 52, the puncture direction 53, and the position of the tip of the puncture instrument 51 are the outputs. As a learner, a stand-alone high-performance computer using a CPU processor and a GPU processor, or a cloud computer is used. Hereinafter, a learning method using a neural network configured by combining perceptrons such as deep learning in the learner will be described. As the learning method, various methods such as random forest, a decision tree, a support vector machine (SVM), logistic regression, a k-nearest neighbor method, and a topic model can be applied.

### (Step S111)

The learner reads a data set that is training data. If it is the first time, the learner reads the first data set, and if it is the i-th time, the learner reads the i-th data set.

### (Step S112)

The learner inputs the input data of the read data set into the neural network.

### (Step S113)

The learner compares an estimation result of the neural network with ground truth data.

### (Step S114)

The learner adjusts a parameter on the basis of a comparison result. The learner adjusts the parameter so as to reduce the difference in the comparison result by executing processing based on back propagation.

### (Step S115)

When the processing for all the data from the first data set to the i-th data set is complete (YES), the learner advances the processing to step S116. When the processing is not complete (NO), the learner returns the processing to step S111, reads the next data set, and repeats the processing in step S111 and subsequent steps.

### (Step S116)

The learner determines whether to continue the learning. When the learning is to be continued (YES), the learner returns the processing to step S111, and again executes, in steps S111 to S115, the processing from the first data set to the i-th data set. When the learning is not to be continued (NO), the learner advances the processing to step S117.

### (Step S117)

The learner stores the machine learning model constructed by the foregoing processing, and ends the processing (end). The storage destination includes an internal memory of the image processing apparatus 20. In the estimation processing described above, the puncture instrument region is estimated using the machine learning model generated in this way.

### <Operations and Effects of Image Processing Apparatus 20 and Ultrasonic Diagnostic Apparatus 1>

In the image processing apparatus 20 and the ultrasonic diagnostic apparatus 1 according to the present embodiment, the training ultrasonic image 50 is input into the machine learning model. This machine learning model is trained on the data set of the training ultrasonic image 50 and the region information corresponding to the two-dimensional region 52. This makes it possible to output an accurate estimation result from the ultrasonic image 60 input into the machine learning model. Therefore, the position of the puncture instrument in the ultrasonic image 60 can be identified more accurately. Hereinafter, this operation and effect will be described.

It is also conceivable to detect a linear brightness distribution similar to the puncture instrument from the ultrasonic image by using image processing techniques and to identify the position of the puncture instrument. However, a plurality of linear brightness distributions similar to the puncture instrument may possibly be present within the ultrasonic image. Therefore, it is difficult to identify the position of the puncture instrument in the ultrasonic image with sufficiently high accuracy by the image processing techniques. In addition, even using the machine learning model, if the ground truth label of the data set does not have sufficient information, there is a possibility that it is difficult to accurately estimate the position at which the puncture instrument is present.

The puncture instrument has a predetermined thickness. However, in a case where the ground truth label is only one-dimensional information corresponding to a region in which the puncture instrument is present in the training ultrasonic image, for example, information in a length direction of the puncture instrument, information in a thickness direction of the puncture instrument is not reflected in the ground truth label. Therefore, even using this machine learning model, there is a possibility that false detection will occur.

In contrast, the machine learning model used by the image processing apparatus 20 is trained on, as the ground truth label, the region information corresponding to the two-dimensional region 52 having a rectangular shape. Thus, information in two directions, i.e., the length direction and the thickness direction of the puncture instrument is learned. When the irradiation of the ultrasonic waves is performed in the thickness direction of the puncture instrument, the portion of the puncture instrument on the side opposite to the side where the irradiation of the ultrasonic waves is performed in the puncture instrument appears with lower brightness. The two-dimensional region 52 can capture such a change in the brightness of the puncture instrument. Therefore, the use of the machine learning model trained using the region information corresponding to the two-dimensional region 52 makes it possible to more accurately estimate the position at which the puncture instrument is present. Consequently, the use of the image processing apparatus 20 makes it possible for the user to more accurately identify the position of the puncture instrument in the ultrasonic image 60.

The data set used to train the machine learning model includes information regarding the puncture direction 53. This makes it possible for the image processing apparatus 20 to estimate the puncture direction of the puncture instrument and the position of the tip of the puncture instrument in the ultrasonic image 60. This makes it possible for the user to perform puncturing on the subject more smoothly.

Furthermore, in the image processing apparatus 20, the machine learning model is stored in the storage 24. This makes it possible to estimate the position of the puncture instrument from the acquired ultrasonic image 60 at a higher processing speed. Therefore, the user can identify the position of the puncture instrument moving inside the body of the subject with a sense closer to real time.

Hereinafter, modification examples of the above-described embodiment will be described. The same reference numerals are given to the same configurations as those of the above-described embodiment, and the description thereof will be omitted.

### First Modification Example

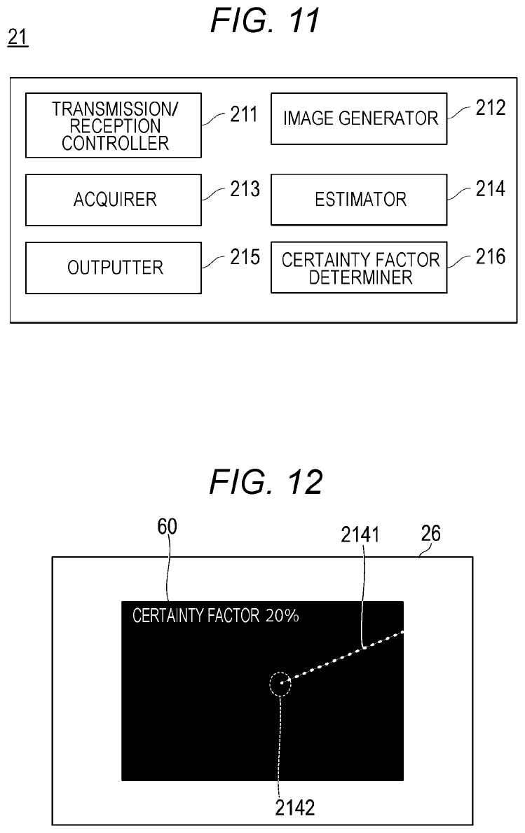

FIG. 11 is a block diagram illustrating a functional configuration of an image processing apparatus 20 according to a first modification example. The image processing apparatus 20 functions as a certainty factor determiner 216 in addition to the transmission/reception controller 211, the image generator 212, the acquirer 213, the estimator 214, and the outputter 215 when the CPU 21 reads a program stored in the storage 24 and executes processing. Except for this point, the image processing apparatus 20 according to the first modification example has the same configuration as the image processing apparatus 20 described in the above embodiment, and achieves the same operation and effect.

The certainty factor determiner 216 determines whether the certainty factor of the puncture instrument region 2141 estimated by the estimator 214 exceeds a predetermined value. The predetermined value is, for example, 80%. When the certainty factor determiner 216 determines that the certainty factor exceeds the predetermined value, the outputter 215 outputs the estimation result in a first mode. When the certainty factor determiner 216 determines that the certainty factor is equal to or less than the predetermined value, the outputter 215 outputs the estimation result in a second mode. The second mode is different from the first mode. The outputter 215 changes the display method of the puncture instrument region 2141 on the display 26 between the first mode and the second mode. The outputter 215 may change the display method of the certainty factor on the display 26 between the first mode and the second mode.

FIG. 12 illustrates an example of the estimation result displayed on the display 26 in the second mode. When the certainty factor determiner 216 determines that the certainty factor is equal to or less than the predetermined value, the outputter 215 causes the display 26 to display the puncture instrument region 2141 with a broken line. When the certainty factor determiner 216 determines that the certainty factor exceeds the predetermined value, the outputter 215 causes the display 26 to display the puncture instrument region 2141 with a solid line as illustrated in FIG. 6.

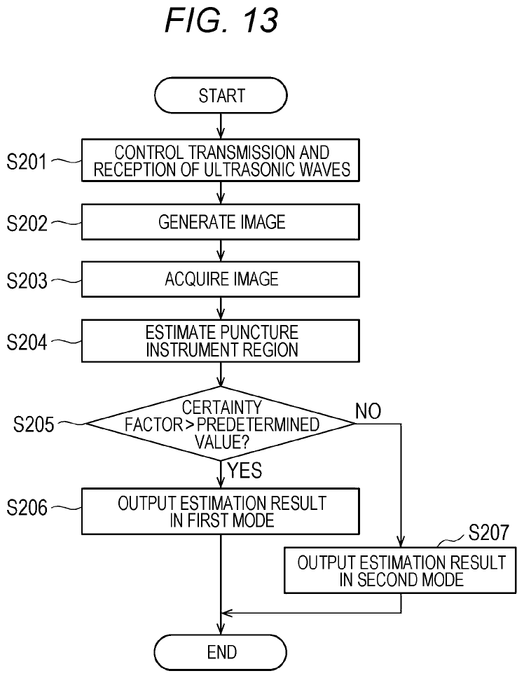

FIG. 13 is a flowchart illustrating a procedure of processing executed by the image processing apparatus 20. The processing of the image processing apparatus 20 illustrated in the flowchart of FIG. 13 is stored as a program in the storage 24 of the image processing apparatus 20 and is executed by the CPU 21 controlling the respective units.

### (Steps S201 to S204)

The image processing apparatus 20 performs processing in steps S201 to S204 in a similar manner to the processing in steps S101 to S104 described above.

### (Step S205)

The image processing apparatus 20 determines whether the certainty factor of the puncture instrument region 2141 estimated in step S204 exceeds the predetermined value.

### (Step S206)

When the image processing apparatus 20 determines, in step S205, that the certainty factor exceeds the predetermined value (step S205: YES), the image processing apparatus 20 outputs the estimation result in the first mode and ends the processing. At this time, the image processing apparatus 20 causes the display 26 to display the puncture instrument region 2141 with a solid line.

### (Step S207)

When the image processing apparatus 20 determines, in step S205, that the certainty factor is equal to or less than the predetermined value (step S205: NO), the image processing apparatus 20 outputs the estimation result in the second mode and ends the processing. At this time, the image processing apparatus 20 causes the display 26 to display the puncture instrument region 2141 with a broken line.

Also in the image processing apparatus 20 according to the first modification example, the training ultrasonic image 50 is input into the machine learning model, as described in the above embodiment. This machine learning model is trained on the data set of the training ultrasonic image 50 and the region information corresponding to the two-dimensional region 52. This makes it possible to output an accurate estimation result from the ultrasonic image 60 input into the machine learning model. Therefore, the position of the puncture instrument in the ultrasonic image 60 can be identified more accurately.

Furthermore, the image processing apparatus 20 outputs the estimation results in different modes in accordance with the certainty factor of the estimated puncture instrument regions 2141. This makes it possible for the user to more intuitively grasp the reliability of the estimated puncture instrument region 2141. This makes it easier for the user to notice the possibility that the puncture instrument is not captured in the acquired ultrasonic image 60.

### Second Modification Example

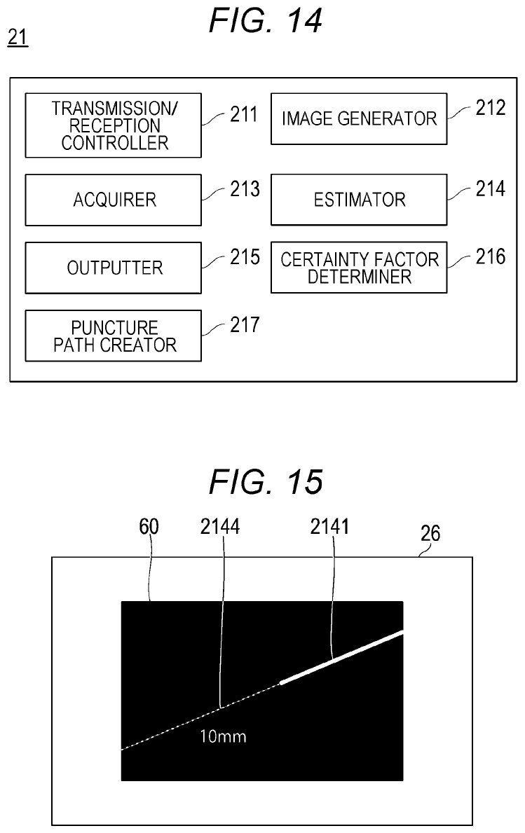

FIG. 14 is a block diagram illustrating a functional configuration of an image processing apparatus 20 according to a second modification example. The image processing apparatus 20 functions as a puncture path creator 217 in addition to the transmission/reception controller 211, the image generator 212, the acquirer 213, the estimator 214, the outputter 215, and the certainty factor determiner 216 when the CPU 21 reads a program stored in the storage 24 and executes processing. Except for this point, the image processing apparatus 20 according to the second modification example has the same configuration as the image processing apparatus 20 described in the first modification example, and achieves the same operation and effect.

The puncture path creator 217 creates a puncture path on the basis of the puncture instrument region 2141 estimated by the estimator 214. The puncture path is a path along which the puncture instrument is predicted to travel in the future on the basis of the current position and direction of the puncture instrument. When the certainty factor of the puncture instrument region 2141 estimated by the estimator 214 exceeds the predetermined value, the puncture path creator 217 creates a puncture path. When the certainty factor of the puncture instrument region 2141 estimated by the estimator 214 is equal to or less than the predetermined value, the puncture path creator 217 does not create a puncture path. The puncture path creator 217 calculates a formula on the basis of the position, the inclination, and the like of the puncture instrument region 2141 and creates a puncture path using the formula.

FIG. 15 illustrates an example of the puncture instrument region 2141 and a puncture path 2144 displayed on the display 26. The puncture path 2144 is indicated by a broken line following the puncture instrument region 2141. The puncture path 2144 may be displayed on the display 26 together with a scale or the like indicating the length.

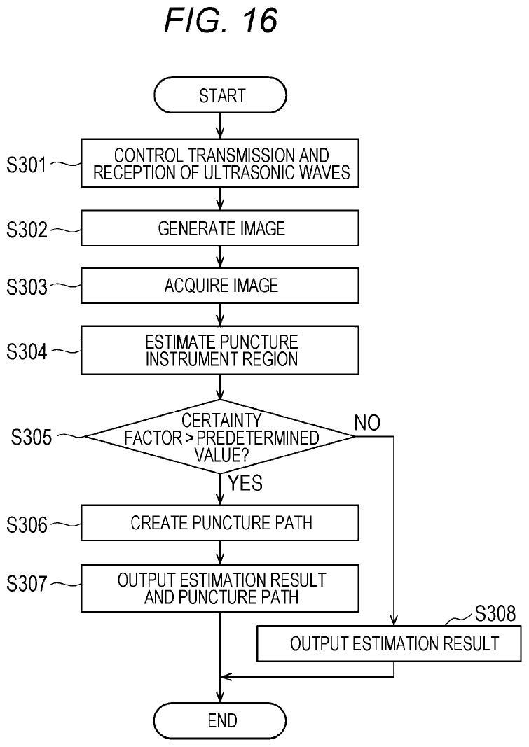

FIG. 16 is a flowchart illustrating a procedure of processing executed by the image processing apparatus 20. The processing of the image processing apparatus 20 illustrated in the flowchart of FIG. 15 is stored as a program in the storage 24 of the image processing apparatus 20 and is executed by the CPU 21 controlling the respective units.

### (Steps S301 to S304)

The image processing apparatus 20 performs processing in steps S301 to S304 in a similar manner to the processing in steps S101 to S104 described above.

### (Step S305)

The image processing apparatus 20 determines whether the certainty factor of the puncture instrument region 2141 estimated in step S304 exceeds the predetermined value.

### (Steps S306 and S307)

When the image processing apparatus 20 determines, in step S305, that the certainty factor exceeds the predetermined value (step S305: YES), the image processing apparatus 20 creates the puncture path 2144 on the basis of the puncture instrument region 2141 estimated in step S304. Thereafter, the image processing apparatus 20 outputs the estimation result and the puncture path 2144, and ends the processing. The image processing apparatus 20 causes the display 26 to display the puncture path 2144 so as to follow the puncture instrument region 2141. At this time, the image processing apparatus 20 may output the estimation result in the first mode.

### (Step S308)

When the image processing apparatus 20 determines, in step S305, that the certainty factor is equal to or less than the predetermined value (step S305: NO), the image processing apparatus 20 outputs the estimation result and ends the processing. At this time, the image processing apparatus 20 may output the estimation result in the second mode.

Also in the image processing apparatus 20 according to the second modification example, the training ultrasonic image 50 is input into the machine learning model, as described in the above embodiment. This machine learning model is trained on the data set of the training ultrasonic image 50 and the region information corresponding to the two-dimensional region 52. This makes it possible to output an accurate estimation result from the ultrasonic image 60 input into the machine learning model. Therefore, the position of the puncture instrument in the ultrasonic image 60 can be identified more accurately.

The image processing apparatus 20 creates the puncture path 2144 on the basis of the estimated puncture instrument region 2141. This makes it possible for the user to predict the future course of the puncture instrument region 2141 and accurately grasp the positional relationship between the tissue and the puncture instrument inside the body of the subject.

The image processing apparatus 20 may create a puncture path regardless of the value of the certainty factor. That is, the image processing apparatus 20 according to the second modification example does not necessarily include the certainty factor determiner 216.

The image processing apparatus 20 may determine whether to create a puncture path on the basis of the position of the puncture instrument region 2141 within the ultrasonic image 60. When the puncture instrument region 2141 is present in the central portion of the ultrasonic image 60, the image processing apparatus 20 determines not to create a puncture path.

### Third Modification Example

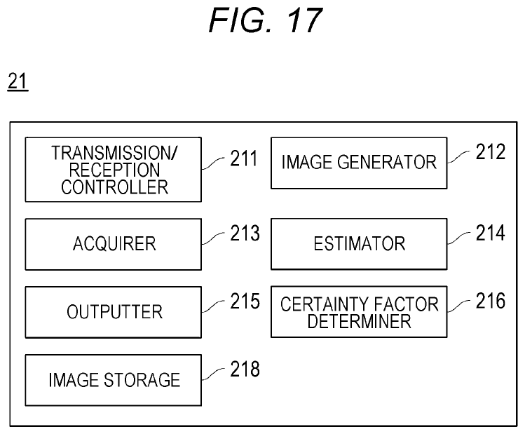

FIG. 17 is a block diagram illustrating a functional configuration of an image processing apparatus 20 according to a third modification example. The image processing apparatus 20 functions as an image storage 218 in addition to the transmission/reception controller 211, the image generator 212, the acquirer 213, the estimator 214, the outputter 215, and the certainty factor determiner 216 when the CPU 21 reads a program stored in the storage 24 and executes processing. Except for this point, the image processing apparatus 20 according to the third modification example has the same configuration as the image processing apparatus 20 described in the above first modification example, and achieves the same operation and effect.

The image storage 218 saves the ultrasonic image 60 acquired by the acquirer 213. The image storage 218 stores the acquired ultrasonic image 60 in the storage 24 or the like, thereby saving the ultrasonic image 60. The image storage 218 may save the ultrasonic image 60 together with the estimation result estimated by the estimator 214. When the certainty factor of the puncture instrument region 2141 estimated by the estimator 214 exceeds the predetermined value, the image storage 218 saves the ultrasonic image 60. When the certainty factor of the puncture instrument region 2141 estimated by the estimator 214 is equal to or less than the predetermined value, the image storage 218 does not save the ultrasonic image 60. The image storage 218 may save the ultrasonic image 60 in an external server or the like.

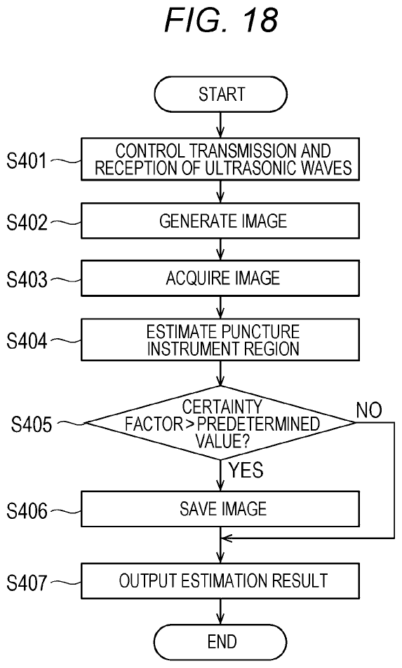

FIG. 18 is a flowchart illustrating a procedure of processing executed by the image processing apparatus 20. The processing of the image processing apparatus 20 illustrated in the flowchart of FIG. 18 is stored as a program in the storage 24 of the image processing apparatus 20 and is executed by the CPU 21 controlling the respective units.

### (Steps S401 to S404)

The image processing apparatus 20 performs processing in steps S401 to S404 in a similar manner to the processing in steps S101 to S104 described above.

### (Step S405)

The image processing apparatus 20 determines whether the certainty factor of the puncture instrument region 2141 estimated in step S404 exceeds a predetermined value. When the image processing apparatus 20 determines, in step S405, that the certainty factor is equal to or less than the predetermined value (step S405: NO), the image processing apparatus 20 proceeds to processing in step S407.

### (Step S406)

When the image processing apparatus 20 determines, in step S405, that the certainty factor exceeds the predetermined value (step S405: YES), the image processing apparatus 20 saves the ultrasonic image 60 acquired in step S401 and proceeds to the processing in step S407. The image processing apparatus 20 may save the ultrasonic image 60 together with the estimation result estimated in step S404.

### (Step S407)

The image processing apparatus 20 outputs the estimation result and ends the processing. The image processing apparatus 20 may execute the processing in step S407 before the processing in step S406, or may execute the processing in step S406 and step S407 at the same time.

Also in the image processing apparatus 20 according to the third modification example, the training ultrasonic image 50 is input into the machine learning model, as described in the above embodiment. This machine learning model is trained on the data set of the training ultrasonic image 50 and the region information corresponding to the two-dimensional region 52. This makes it possible to output an accurate estimation result from the ultrasonic image 60 input into the machine learning model. Therefore, the position of the puncture instrument in the ultrasonic image 60 can be identified more accurately.

Furthermore, in the image processing apparatus 20, the ultrasonic image 60 is automatically saved. This makes it possible to facilitate the saving of the ultrasonic image 60 even when both hands of the user are full at the time of the puncturing. Therefore, the saved ultrasonic image 60 can be used for analysis or the like of the state of the subject.

The image processing apparatus 20 may save the ultrasonic image 60 regardless of the value of the certainty factor. That is, the image processing apparatus 20 according to the third modification example does not necessarily include the certainty factor determiner 216. The image processing apparatus 20 according to the third modification example may further include the puncture path creator 217.

The image processing apparatus 20 may determine whether to save the ultrasonic image 60 on the basis of the position of the puncture instrument region 2141 within the ultrasonic image 60. When the puncture instrument region 2141 is present in the central portion of the ultrasonic image 60, the image processing apparatus 20 determines to save the ultrasonic image 60. Alternatively, the image processing apparatus 20 may determine whether to save the ultrasonic image 60 on the basis of the positional relationship with the tissue inside the body of the subject. When the puncture instrument region 2141 is present in the vicinity of a nerve inside the body of the subject, the image processing apparatus 20 determines to save the ultrasonic image 60.

### Fourth Modification Example

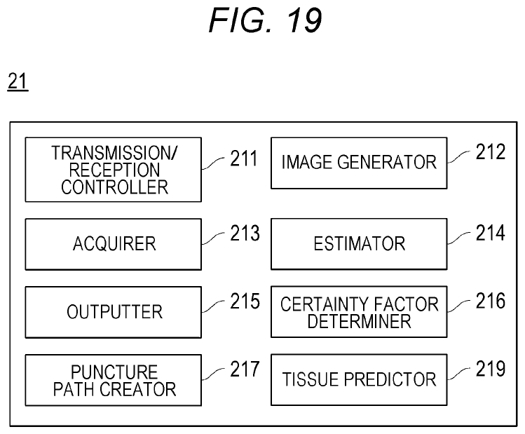

FIG. 19 is a block diagram illustrating a functional configuration of an image processing apparatus 20 according to a fourth modification example. The image processing apparatus 20 functions as a tissue predictor 219 in addition to the transmission/reception controller 211, the image generator 212, the acquirer 213, the estimator 214, the outputter 215, the certainty factor determiner 216, and the puncture path creator 217 when the CPU 21 reads a program stored in the storage 24 and executes processing. Except for this point, the image processing apparatus 20 according to the fourth modification example has the same configuration as the image processing apparatus 20 according to the second modification example, and achieves the same operation and effect.

The tissue predictor 219 predicts a target tissue present within the ultrasonic image 60 on the basis of the ultrasonic image 60 acquired by the acquirer 213. The target tissue is a specific blood vessel, a nerve, or the like present inside the body of the subject. The tissue predictor 219 predicts a target tissue present within the ultrasonic image 60, using the machine learning model. This machine learning model is trained on a data set of a training ultrasonic image in which at least a part of the target tissue is captured and region information corresponding to a two-dimensional region in which the target tissue is present in this training ultrasonic image. The target tissue is, for example, a blood vessel or a nerve.

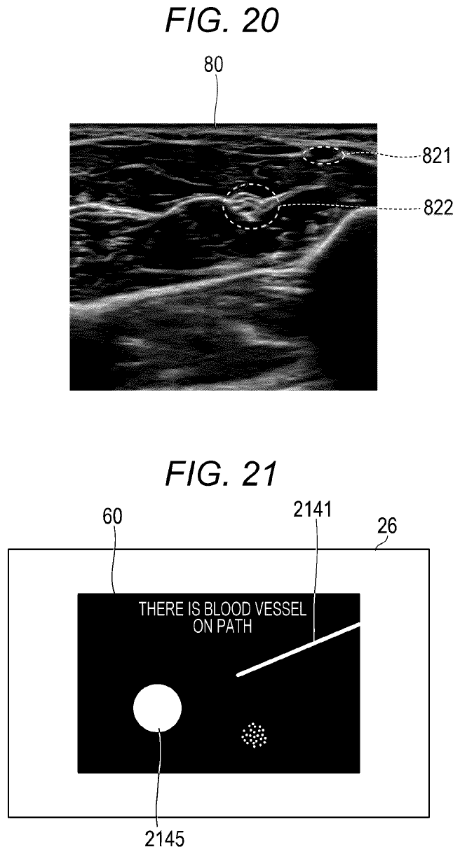

FIG. 20 illustrates an example of a training ultrasonic image 80 in which blood vessels and nerves are captured. The training ultrasonic image 80 includes a two-dimensional region 821 in which a blood vessel is present and a two-dimensional region 822 in which a nerve is present. The machine learning model is trained on a data set of the training ultrasonic image 80 and the region information corresponding to the two-dimensional regions 821 and 822.

When the tissue predictor 219 has predicted that the target tissue is present within the ultrasonic image 60, the outputter 215 outputs information regarding the target tissue together with the estimation result.

When the distance between the tissue and the puncture instrument region 2141 is equal to or less than a predetermined value, the outputter 215 may output alert information for calling the user's attention. The outputter 215 outputs alert information by causing the display 26 to display a message, an icon, or the like for calling attention. On the display 26, a message, an icon, or the like may be displayed so as to be superimposed on the ultrasonic image 60, or a message, an icon, or the like may be displayed outside the ultrasonic image 60. The outputter 215 may output alert information by causing the audio inputter/outputter 28 to output a warning sound, a warning message, or the like.

FIG. 21 illustrates an example of the estimation result and the tissue displayed on the display 26. The puncture instrument region 2141 and a blood vessel 2145 within the ultrasonic image 60 are displayed on the display 26. The display 26 further displays a message for urging the user to pay attention to the blood vessel 2145.

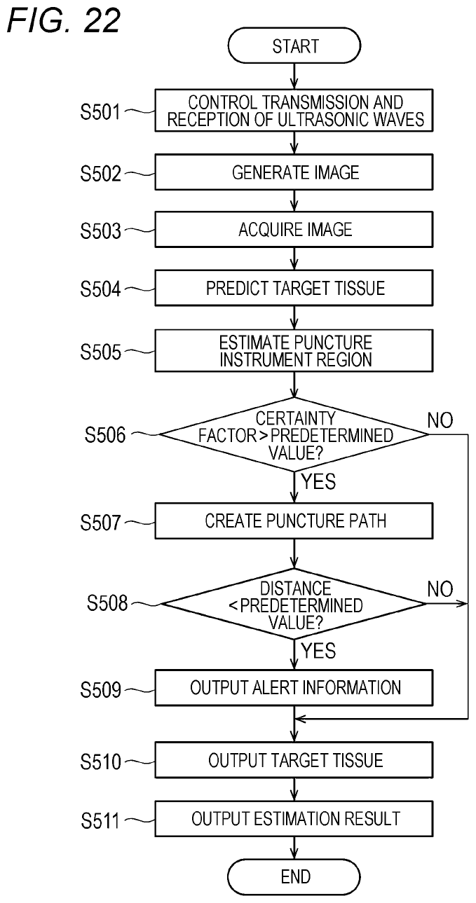

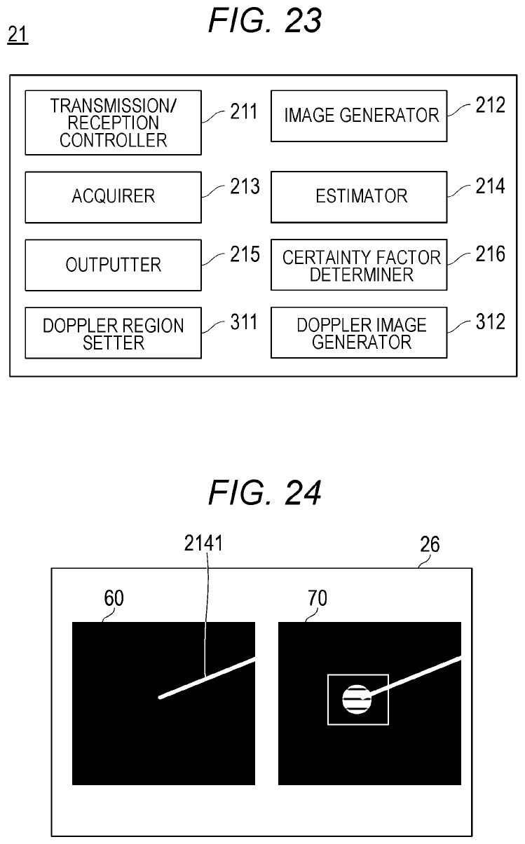

FIG. 22 is a flowchart illustrating a procedure of processing executed by the image processing apparatus 20. The processing of the image processing apparatus 20 illustrated in the flowchart of FIG. 22 is stored as a program in the storage 24 of the image processing apparatus 20 and is executed by the CPU 21 controlling the respective units.

### (Steps S501 to S503)

The image processing apparatus 20 performs processing in steps S501 to S503 in a similar manner to the processing in steps S101 to S103 described above.

### (Step S504)

The image processing apparatus 20 predicts a target tissue present within the ultrasonic image 60 on the basis of the ultrasonic image 60 acquired in step S503.

### (Step S505)

The image processing apparatus 20 estimates the puncture instrument region 2141 by inputting the ultrasonic image 60 acquired in step S503 into the machine learning model. At this time, the image processing apparatus 20 calculates the certainty factor of the puncture instrument region 2141. The image processing apparatus 20 may execute the processing in step S505 before the processing in step S504, or may execute the processing in step S504 and step S505 at the same time.

### (Step S506)

The image processing apparatus 20 determines whether the certainty factor of the puncture instrument region 2141 estimated in step S505 exceeds the predetermined value.

### (Step S511)

When the image processing apparatus 20 determines, in step S506, that the certainty factor is equal to or less than the predetermined value (step S506: NO), the image processing apparatus 20 outputs the estimation result and ends the processing.

### (Step S507)

When the image processing apparatus 20 determines, in step S506, that the certainty factor exceeds the predetermined value (step S506: YES), the image processing apparatus 20 creates the puncture path 2144 on the basis of the puncture instrument region 2141 estimated in step S505.

### (Step S508)

The image processing apparatus 20 determines whether the distance between the puncture path 2144 created in step S507 and the puncture instrument region 2141 estimated in step S505 is less than the predetermined value.

### (Step S510 and S511)

When the image processing apparatus 20 determines, in step S508, that the distance between the puncture path 2144 and the puncture instrument region 2141 is equal to or greater than the predetermined value (step S508: NO), the image processing apparatus 20 outputs the target tissue and the estimation result and ends the processing.

### (Step S509, S510, and S511)

When the image processing apparatus 20 determines, in step S508, that the distance between the puncture path 2144 and the puncture instrument region 2141 is less than the predetermined value (step S508: YES), the image processing apparatus 20 outputs alert information, the target tissue, and the estimation result and ends the processing.

Also in the image processing apparatus 20 according to the fourth modification example, the training ultrasonic image 50 is input into the machine learning model, as described in the above embodiment. This machine learning model is trained on the data set of the training ultrasonic image 50 and the region information corresponding to the two-dimensional region 52. This makes it possible to output an accurate estimation result from the ultrasonic image 60 input into the machine learning model. Therefore, the position of the puncture instrument in the ultrasonic image 60 can be identified more accurately.

Furthermore, since the image processing apparatus 20 predicts the target tissue captured in the ultrasonic image 60, the user can easily grasp the positional relationship between the puncture instrument and the target tissue inside the body of the subject. In addition, since the image processing apparatus 20 outputs the alert information when the distance between the target tissue and the puncture instrument is short, it is possible to call the user's attention.

The image processing apparatus 20 may create the puncture path 2144 regardless of the value of the certainty factor. That is, the image processing apparatus 20 according to the fourth modification example does not necessarily include the certainty factor determiner 216. Alternatively, the image processing apparatus 20 does not necessarily create a puncture path. That is, the image processing apparatus 20 according to the fourth modification example does not necessarily include the puncture path creator 217. The image processing apparatus 20 according to the fourth modification example may further include the image storage 218.

### Fifth Modification Example

FIG. 23 is a block diagram illustrating a functional configuration of an image processing apparatus 20 according to a fifth modification example. The image processing apparatus 20 functions as a Doppler region setter 311 and a Doppler image generator 312 in addition to the transmission/reception controller 211, the image generator 212, the acquirer 213, the estimator 214, the outputter 215, and the certainty factor determiner 216 when the CPU 21 reads a program stored in the storage 24 and executes processing. Except for this point, the image processing apparatus 20 according to the fifth modification example has the same configuration as the image processing apparatus 20 described in the above first modification example, and achieves the same operation and effect.

The Doppler region setter 311 sets a Doppler region within the ultrasonic image 60 on the basis of the puncture instrument region 2141 estimated by the estimator 214. The Doppler region is a region in which a Doppler image is generated.

The Doppler image generator 312 generates a Doppler image on the basis of Doppler signals generated in the Doppler region. The Doppler region is set by the Doppler region setter 311. The Doppler signals are generated by the ultrasonic probe 10 irradiating the Doppler region with ultrasonic waves.

FIG. 24 illustrates an example of a Doppler image 70 displayed on the display 26. The Doppler image 70 is displayed side by side with the ultrasonic image 60. The puncture instrument region 2141 is displayed in the ultrasonic image 60. A tissue present within the ultrasonic image 60 is captured in the Doppler image 70. Liquid medicine or the like present within the ultrasonic image 60 may be captured in the Doppler image 70.

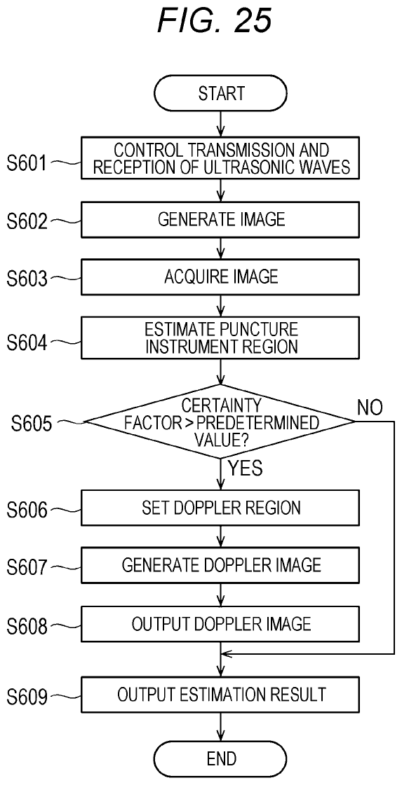

FIG. 25 is a flowchart illustrating a procedure of processing executed by the image processing apparatus 20. The processing of the image processing apparatus 20 illustrated in the flowchart of FIG. 25 is stored as a program in the storage 24 of the image processing apparatus 20 and is executed by the CPU 21 controlling the respective units.

### (Steps S601 to S604)

The image processing apparatus 20 performs processing in steps S601 to S604 in a similar manner to the processing in steps S101 to S104 described above.

### (Step S605)

The image processing apparatus 20 determines whether the certainty factor of the puncture instrument region 2141 estimated in step S604 exceeds the predetermined value.

### (Step S609)

When the image processing apparatus 20 determines, in step S605, that the certainty factor is equal to or less than the predetermined value (step S605: NO), the image processing apparatus 20 outputs the estimation result and ends the processing.

### (Step S606)

When the image processing apparatus 20 determines, in step S605, that the certainty factor exceeds the predetermined value (step S605: YES), the image processing apparatus 20 sets a Doppler region within the ultrasonic image 60.

### (Step S607)

The image processing apparatus 20 generates a Doppler image by irradiating the Doppler region set in step S606 with ultrasonic waves.

### (Step S608 and S609)

The image processing apparatus 20 outputs the Doppler image generated in step S608 together with the estimation result, and ends the processing.

Also in the image processing apparatus 20 according to the fifth modification example, the training ultrasonic image 50 is input into the machine learning model, as described in the above embodiment. This machine learning model is trained on the data set of the training ultrasonic image 50 and the region information corresponding to the two-dimensional region 52. This makes it possible to output an accurate estimation result from the ultrasonic image 60 input into the machine learning model. Therefore, the position of the puncture instrument in the ultrasonic image 60 can be identified more accurately.

In the image processing apparatus 20, the Doppler region is automatically set. This makes it possible to facilitate the generation of the Doppler image even when both hands of the user are full at the time of the puncturing. Therefore, the user can easily grasp the positional relationship between the puncture instrument and the tissue inside the body of the subject.

The image processing apparatus 20 may generate a Doppler image regardless of the value of the certainty factor. That is, the image processing apparatus 20 according to the fifth modification example does not necessarily include the certainty factor determiner 216. The image processing apparatus 20 according to the fifth modification example may further include at least one of the puncture path creator 217, the image storage 218, and the tissue predictor 219.

The present invention is not limited to the above-described embodiment and the modification examples, and various modifications can be made within the scope of the claims.

The ultrasonic probe 10 and the image processing apparatus 20 each may include any constituent elements other than the above-described constituent elements, or may not include some of the above-described constituent elements. The image processing apparatus 20 may be, for example, an information processing apparatus as long as it has an information processing function for outputting an estimation result on the basis of the ultrasonic image 60.

Each of the ultrasonic probe 10 and the image processing apparatus 20 may be configured by a plurality of apparatuses or may be configured by a single apparatus. Furthermore, the function of each component may be implemented by another component. Some or all of the functions of the image processing apparatus 20 may be implemented by the ultrasonic probe 10, an external server, or the like. The machine learning model may be stored in an external server or the like.

Furthermore, the two-dimensional region 52 may have a shape other than a rectangular shape. The two-dimensional region 52 may have a shape such as an ellipse.

Furthermore, the processing units of the flowcharts in the above embodiment and the modification examples are divided in accordance with the main processing contents for easy understanding of each processing. The present invention is not limited by how the processing steps are classified. Each processing can be further divided into more processing steps. In addition, a single processing step may execute more processing.

Means and methods for performing various kinds of processing in the system according to the above-described embodiment can be implemented by either a dedicated hardware circuit or a programmed computer. The above program may be provided by a non-transitory computer-readable storage medium such as a flexible disk or a CD-ROM, or may be provided online via a network such as the Internet. In this case, the programs recorded on the non-transitory computer-readable storage medium are usually transferred to and stored in a storage unit such as a hard disk. Furthermore, the above program may be provided as independent application software, or may be incorporated, as one function of a system, in software of a device in the system.

Although embodiments of the present invention have been described and illustrated in detail, the disclosed embodiments are made for purpose of illustration and example only and not limitation. The scope of the present invention should be interpreted by terms of the appended claims.

What is claimed is:

1. An information processing apparatus comprising a hardware processor that:
acquires an ultrasonic image of a subject into which a needle-shaped instrument has been inserted; and
outputs an estimation result that is output by inputting the ultrasonic image acquired into a machine learning model, wherein
the machine learning model is trained on a data set of a training ultrasonic image in which at least a part of the needle-shaped instrument is captured and region information corresponding to a two-dimensional region in which the needle-shaped instrument is present in the training ultrasonic image.

2. The information processing apparatus according to claim 1, wherein the hardware processor estimates a needle-shaped instrument region in which the needle-shaped instrument is present in the ultrasonic image by inputting the ultrasonic image acquired into the machine learning model, and
outputs the estimation result including information regarding the needle-shaped instrument region estimated.

3. The information processing apparatus according to claim 2, wherein the estimation result to be output further includes information regarding a certainty factor of the needle-shaped instrument region estimated.

4. The information processing apparatus according to claim 3, further comprising a notificator that notifies a user of the estimation result that has been output, wherein
the notificator is configured to be able to change a mode of notification to the user in accordance with the certainty factor.

5. The information processing apparatus according to claim 2, wherein the estimation result to be output further includes information regarding a position at which a tip of the needle-shaped instrument is present in the needle-shaped instrument region estimated.

6. The information processing apparatus according to claim 1, wherein the data set further includes information regarding a traveling direction of the needle-shaped instrument in the training ultrasonic image.

7. The information processing apparatus according to claim 6, wherein a tip of the needle-shaped instrument is present in the two-dimensional region.

8. The information processing apparatus according to claim 6, wherein
the two-dimensional region has a rectangular shape, and
the needle-shaped instrument is disposed on a diagonal line of the rectangular shape.

9. The information processing apparatus according to claim 6, wherein
the training ultrasonic image is a B-mode image, and
the two-dimensional region contains portions having mutually-different degrees of brightness.

10. The information processing apparatus according to claim 1, further comprising a memory in which the machine learning model is stored.

11. The information processing apparatus according to claim 1, wherein the two-dimensional region is continuously provided.

12. An ultrasonic diagnostic apparatus comprising:
a probe that irradiates, with an ultrasonic wave, a subject into which a needle-shaped instrument has been inserted, and receives the ultrasonic wave reflected off the subject; and
a hardware processor that generates an ultrasonic image of the subject on a basis of the ultrasonic wave,
acquires the ultrasonic image generated, and
outputs an estimation result that is output by inputting the ultrasonic image acquired into a machine learning model, wherein
the machine learning model is trained on a data set of a training ultrasonic image in which at least a part of the needle-shaped instrument is captured and region information corresponding to a two-dimensional region in which the needle-shaped instrument is present in the training ultrasonic image.

13. An information processing method that is executed by an information processing apparatus, the information processing method comprising:
acquiring an ultrasonic image of a subject into which a needle-shaped instrument has been inserted; and
outputting an estimation result that is output by inputting the ultrasonic image acquired into a machine learning model, wherein
the machine learning model is trained on a data set of a training ultrasonic image in which at least a part of the needle-shaped instrument is captured and region information corresponding to a two-dimensional region in which the needle-shaped instrument is present in the training ultrasonic image.

14. A non-transitory computer-readable storage medium storing an information processing program that causes a computer to execute the information processing method according to claim 13.
